(19) United States
(12) Patent Application Publication
(10) Pub. No.: US 2009/0232912 A1
(43) Pub. Date: Sep. 17, 2009

(54) Method For Preparing Extract For The Prevention and Treatment Of Hyperlipidemia and Obesity From the Extract of Aster Spathulifolius Aerial Part and Composition Containing The Same

(76) Inventor: Se Young Chung, Seoul (KR)

Correspondence Address: SUGHRUE MION, PLLC, 2100 PENNSYLVANIA AVENUE, N.W., SUITE 800, WASHINGTON, DC, 20037, US

(21) Appl. No.: 11/909,109

(22) PCT Filed: Mar. 20, 2006
(86) PCT No.: PCT/KR06/01003

§ 371 Date: Sep. 19, 2007

(30) Foreign Application Priority Data
Mar. 19, 2005 (KR) 10-2005-0022965

(51) Int. Cl. A61K 36/00; A61K 31/01

(52) U.S. Cl. 424/725; 514/762

(57) ABSTRACT

The present invention relates to a method for preparing an extract of the aerial part of Aster spathulifolius effective in preventing and treating hyperlipidemia and obesity and a composition comprising the extract. Since most of salt which is originally abundant in Aster spathulifolius, phenolic compounds which are unstable and susceptible to oxidation, and mucous substances are simply removed by the preparation method in accordance with the present invention, the present invention can significantly increase the content of terpenes, or the active ingredient, and reduce production cost. The composition of the present invention can be used as functional food for aiding the prevention and treatment of hyperlipidemia- and obesity-related arteriosclerosis, cardiovascular disease, cerebral thrombosis, liver disease, thrombosis, diabetes, etc.

### TECHNICAL FIELD

The present invention relates to a method for preparing extract for the prevention and treatment of hyperlipidemia and obesity from the extract of the aerial part of Aster spathulifolius and a composition containing the same. Since the extract of the aerial part of Aster spathulifolius is effective in preventing hyperlipidemia and obesity, it can be utilized in compositions or functional foods for preventing such diseases as arteriosclerosis, cardiovascular disease, cerebral thrombosis, liver disease, thrombosis and hyperlipidemia or for aiding the treatment of the diseases.

### BACKGROUND ART

Obesity occurs when energy uptake exceeds energy consumption, leading to accumulation of excess calories in adipose tissues and long-term metabolic imbalance. Obesity is a risk factor for such adult diseases as hyperlipidemia, hypertension, arthritis, cholelithiasis, diabetes, myocardial infarction, breast cancer and fatty liver.

In Korea, uptake of calories as fat increased from 7.3% in 1970 to 18.8% in 1995 and is expected to have reached 25% in 2005. With the increase in fat uptake, various chronic diseases including obesity, stroke, arteriosclerosis, hypertension and diabetes have increased. Particularly, cardiovascular diseases increased significantly.

According to the 2001 data published by the Korea National Statistical Office, the main causes of death was cancer, cerebrovascular disease, heart disease, diabetes and liver disease. Particularly, diseases of the circulatory system such as cardiovascular disease, cerebrovascular disease, arteriosclerosis and thrombosis are emerging as main causes of death. Considering that 42% of the people who died of the diseases of the circulatory system were overweight, obesity seems to be closely related with adult diseases.

The increase in uptake of calories and fats seems to have increased in the blood cholesterol level, resulting in accelerated plaque accumulation in arteries and increased outbreak of cardiovascular disease. Accordingly, a lot of researches are underway on medicines or natural foods that can reduce lipid blood level. As functional foods having physiological activities are gaining focus, researches are actively in progress for finding out plant materials effective in preventing obesity.

Aster spathulifolius is a perennial herb growing along the coasts. It belongs to the chrysanthemum family, Campanulales order, Dicotyledon division. Its stem is rather woody and has a lot of branches. It grows aslant to be about 30-60 cm tall. Thick, reversed oval-shaped leaves are arranged alternately. At the lower part of the stem, the leaves bud out in large numbers. The leaves look white, with hairs densely packed on both sides. The edge of the leaf is smooth or emarginated and looks like a spatula. Light purple flowers bloom from July to November at the tip of the branches. Involucres are semispherical and lenticels are hairy and arranged in three rows. Fruits ripe in November and pappi are light brown and have bristles. There are no scientific researches on the medicinal effect of Aster spathulifolius, but young leaves of the plant have been used as food and the whole plant has been used to treat diabetes, cystitis, etc. as folk remedies. It is also grown as dwarfed tree in a pot because of its winter-surviving tenacity and woody characteristics (Doosan World Encyclopedia EnCyber).

Terpene glycosides of the whole plant of Aster spathulifolius, including labda-7,14-dien-13(R)-ol-4-O-acetyl-α-L-6-deoxyidopyranoside and labda-7,14-dien-13(R)-ol-α-L-6 deoxyidopyranoside and some pigments of its flower were identified. But, there is little research on other ingredients of Aster spathulifolius.

### DISCLOSURE OF THE INVENTION

### Technical Problem

The present inventors paid attention to the fact that the aerial part (and the whole plant) of Aster spathulifolius is used to treat diabetes as folk remedies. While isolating the ingredients of Aster spathulifolius, they confirmed that the Aster spathulifolius extract is effective in preventing and treating hyperlipidemia and obesity in high fat diet-induced obese rats.

An object of the present invention is to provide a method for preparing the extract of the aerial part of Aster spathulifolius effective in preventing and treating hyperlipidemia and obesity and a composition and functional food for preventing and treating cardiovascular diseases and hyperlipidemia comprising the extract of the aerial part of Aster spathulifolius as active ingredient.

### Technical Solution

The present invention provides a method for preparing the extract of the aerial part of Aster spathulifolius (extract of Aster spathulifolius aerial part, EASA or AE-B) comprising the steps of: washing the dried powder of the aerial part of Aster spathulifolius with water at room temperature and drying the same; and dissolving the powder of the aerial part of Aster spathulifolius in a solvent to obtain an extract.

The present invention also provides a composition for preventing and treating hyperlipidemia and obesity comprising a terpene compound extracted from the aerial part of Aster spathulifolius.

The present invention also provides a functional health food for preventing and treating hyperlipidemia, obesity and cardiovascular diseases, which comprises the composition for preventing and treating hyperlipidemia and obesity in association with a sitologically acceptable carrier, diluent or excipient.

The present invention also provides an use of the extract of the aerial part of Aster spathulifolius comprising the terpene compound for preventing and treating hyperlipidemia, obesity and cardiovascular diseases.

According to analysis, the extract of the aerial part of Aster spathulifolius contains at least 80% of terpene compounds, including germacron, α-spinasterol and its glycoside, α- and β-amyrin and labdadienol. It was confirmed that the extract of the aerial part of Aster spathulifolius which contains such terpene compounds is effective in preventing and treating hyperlipidemia and obesity. Thus, the extract of the aerial part of Aster spathulifolius can be used as functional food for aiding the prevention and treatment of hyperlipidemia- and obesity-related arteriosclerosis, cardiovascular disease, cerebral thrombosis, liver disease, thrombosis, diabetes, etc.

Hereunder is given a more detailed description on the method for preparing the extract of the aerial part of Aster spathulifolius and the composition for preventing and treating hyperlipidemia and obesity and the use thereof.

The method for preparing the extract of the aerial part of Aster spathulifolius in accordance with the present invention comprises the steps of: washing the dried powder of the aerial part of Aster spathulifolius with water at room temperature and drying the same; and dissolving the powder of the aerial part of Aster spathulifolius in a solvent to obtain an extract.

More particularly, the washing in the first step may be carried out by washing the powder of the aerial part of Aster spathulifolius dried at room temperature with water of about 5 to 20 times, preferably about 10 to 15 times, the weight of the powder at room temperature to 100° C., preferably at room temperature, for about 1 to 12 hours, preferably for 2 to 3 hours, by reflux, ultrasonification, percolation, etc. Especially, reflux washing is preferred and the washing process is repeated for 1 to 5 times, preferably for 2 to 4 times.

And, 5 to 20 parts by weight of alcohol, a mixture solvent of water and alcohol (water/alcohol=˜3:7 to 1:20 by volume) or a mixture solvent of n-hexane and alcohol (n-hexane/alcohol=˜1:1), per 100 parts by weight of the powder of the aerial part of Aster spathulifolius washed and dried in the first step, may be used as the solvent in the second step. Preferably, the alcohol is 90% ethanol.

In the second step, an extract of the aerial part of Aster spathulifolius is obtained using the solvent at room temperature to 100° C., preferably at about 85 to 100° C., by reflux cooling for 1 to 12 hours, preferably for 2 to 5 hours, or by percolation for 1 to 7 days. Preferably, the extraction process is repeated for 1 to 5 times, preferably for 3 to 4 times.

Following the second step, it is preferred to carry out a treatment process (third step) for the extract, including filtering, concentration under reduced pressure and drying, in order to obtain a purified product.

Substances that do not contribute to the prevention or treatment of hyperlipidemia and obesity, including caffeoylquinic acid, derivatives thereof, tannin, salt, etc., are mostly removed during the second and third steps. The extract obtained in the second step may be utilized as an active ingredient for preventing and treating hyperlipidemia and obesity.

The resultant extraction product is characterized by having an increased content of the ingredients effective in preventing and treating hyperlipidemia and obesity and a less content of easily deteriorated compounds and such ingredients as salt that may cause adverse reactions without contributing to the prevention or treatment of hyperlipidemia and obesity. In particular, the extraction product of Aster spathulifolius obtained by the above method is effective in preventing and treating hyperlipidemia and obesity, which seems to be because of the terpene compounds isolated from Aster spathulifolius.

Terpene compounds as mentioned herein include germacron (hereunder abbreviated as AE-1), α-spinasterol-O-β-D-glucopyranoside (hereunder abbreviated as ABP), α- and β-spinasterol, α- and β-amyrin, labdane terpenes such as labda-7,14-dien-13(R)-ol-β-fucopyranoside (novel compound), etc. The terpene compounds account for 80.0-99.6 wt % of the extract of Aster spathulifolius.

In addition to the terpene compounds, 4,5-dicaffeoylquiic acid and methyl 4,5-dicaffeoylquinate, which are caffeoylquinate compounds, may be included in less than 0.3%. And, salt, which may cause adverse effect, is included in less than 0.1%.

As will be described later, the extract of the aerial part of Aster spathulifolius is appropriate for use as active ingredient of the composition for preventing and treating hyperlipidemia and obesity. The effect of preventing and treating hyperlipidemia and obesity can be attained when at least one terpene compound extracted from the aerial part of Aster spathulifolius, which is selected from the group consisting of germacron; α-spinasterol and glycoside thereof; α- or β-amyrin; and labdadienol glycoside is included.

In another embodiment of the present invention, the present invention provides a functional health food for preventing hyperlipidemia and obesity, which comprises the extract of the aerial part of Aster spathulifolius obtained by the afore-mentioned method as active ingredient in association with a sitologically acceptable carrier, diluent, excipient or aromatic.

The extract of the aerial part of Aster spathulifolius prepared by the present invention was confirmed to be effective in improving hyperlipidemia and obesity in high fat diet-induced obese animals. This implies that the extract of the aerial part of Aster spathulifolius can be used as functional food for preventing and treating such adult diseases as hyperlipidemia, hypertension, arthritis, cholelithiasis, diabetes, myocardial infarction, fatty liver, etc., which result from long-term metabolic imbalance caused by accumulation of excess calories in fat tissues.

And, since the extract of the aerial part of Aster spathulifolius is free from toxicity or side effects, it can be safely used for a long period of time for preventive purpose.

In the functional food for improving obesity and hyperlipidemia in accordance with the present invention, the extract of the aerial part of Aster spathulifolius is preferably comprised in 0.1-60 wt % based on the total weight of the composition. If the content of the extract of the aerial part of Aster spathulifolius is smaller, the effect of improving obesity and hyperlipidemia is insufficient. In contrast, if the content is larger, a solubility problem may occur. In addition, the effect of improving obesity and hyperlipidemia does not increase significantly by further addition of the extract.

Examples of the carrier, excipient or diluent that can be used in the functional food of the present invention are lactose, dextrose, sucrose, sorbitol, mannitol, xylitol, erythritol, maltitol, starch, acacia gum, alginate, gelatin, calcium phosphate, calcium silicate, cellulose, methylcellulose, amorphous cellulose, polyvinylpyrrolidone, water, methylhydroxybenzoate, propylhydroxybenzoate, talc and magnesium stearate.

The functional food may be prepared into an oral administration form, including powder, granule, tablet, capsule, suspension, emulsion, syrup, etc., by conventional methods. Particularly, a tablet form is preferable.

A diluent or excipient such as filler, expander, binder, wetting agent, disintegrator, surfactant, etc. may be added to the preparation. Solid preparations for oral administration including tablet, pill, powder, granule, capsule, etc. may comprise, in addition to the extract of the aerial part of Aster spathulifolius, at least one excipient, for example, starch, calcium carbonate, sucrose, lactose, gelatin, etc.

Besides simple excipients, lubricants such as magnesium stearate and talc may be added. Liquid preparations for oral administration including suspension, internal medicine, emulsion, syrup, etc. may comprise various excipients, for example, wetting agent, sweetener, aromatic, preservative, etc., as simple diluent. Non-oral preparations include sterilized aqueous solution, non-aqueous solution, suspension, emulsion, freeze dried preparation and suppository.

Although changeable depending on the age, sex or body weight of the taker, the general dosage of the extract of the aerial part of Aster spathulifolius in accordance with the present invention is 0.01 to 500 mg/kg, preferably 0.1 to 100 mg/kg, a day. It may be administered at once or as split into several portions. Of course, the administration dosage of the extract of the aerial part of Aster spathulifolius may be increased or decreased depending on the administration route, severity of disease, sex, body weight, age, etc. Thus, the afore-mentioned administration dosage does not limit the scope of the present invention in any aspect.

The functional food of the present invention may be given to mammals, including rat, mouse, livestock and human. Since the extract of Aster spathulifolius of the present invention is free from toxicity or side effects, it can be safely used for a long period of time for preventive purpose.

### ADVANTAGEOUS EFFECTS

The extract of the aerial part of Aster spathulifolius in accordance with the present invention has been confirmed to be effective in preventing and treating hyperlipidemia and obesity in rats fed a high fat diet. It significantly reduced body fat weight, hyperlipidemia and hepatic lipid level, total cholesterol, free fatty acid, serum leptin level, insulin, etc. and promoted the expression of the mRNA's of the PPARγ and UCP genes. The method for preparing the extract of the aerial part of Aster spathulifolius in accordance with the present invention is a very simple and economical method. Also, the content of terpene, or the active ingredient, can be increased significantly because most of salt, which is included in Aster spathulifolius in large content, phenolic compounds, which are unstable and susceptible to oxidation, and mucous substances are removed. The composition comprising the extract of the aerial part of Aster spathulifolius as active ingredient in accordance with the present invention can be utilized in functional foods for aiding the prevention and treatment of hyperlipidemia- and obesity-related cardiovascular disease, arteriosclerosis, cerebral thrombosis, liver disease, thrombosis, diabetes, etc.

### BRIEF DESCRIPTION OF THE DRAWINGS

FIG. 1 illustrates the process of isolating the active ingredient from the extract of the aerial part of Aster spathulifolius.

FIG. 2 shows the white adipocytes obtained in Test Example 4.

FIG. 3 shows the magnetic resonance images (MRI) of abdominal fat and visceral fat in Test Example 4.

FIG. 4 shows the total serum triglyceride (TG), total cholesterol (TC), HDL-cholesterol (HDL-C) levels and the ratio of HDL-C/TC in Test Example 4.

FIG. 5 shows the magnetic resonance images (MRI) of abdominal fat and visceral fat in Test Example 5.

FIG. 6 shows the hepatic UCP2 mRNA expression level in Test Example 5.

FIG. 7 shows the hepatic PPARγ mRNA expression level in Test Example 5.

### BEST MODE FOR CARRYING OUT THE INVENTION

### Example 1

### Preparation of the Extract of Aster spathulifolius

Washing of Aerial Part of Aster spathulifolius

Aster spathulifolius (collected in Jeju Island, Korea in October to December) was dried, powdered and used after being filtered through a #4 sieve. 10 kg of the dried powder was suspended in 100 L of water and the resulting suspension was shaken and filtered and then used for the preparation of extract of the invention. The washing procedure was repeated twice until the concentration of chloride in the filtrate was less than 10 ppm.

Preparation of Extract of Aerial Part of Aster spathulifolius

The above washed powder of Aster spathulifolius was suspended in 100 L of aqueous ethanol (H₂O/ethanol=1:10), heated at of 85-100° C. for 2-5 hours, and filtered through filter paper. Such extraction was conducted two more times, and the combined filtrate was concentrated using a rotary evaporator at 40° C. to obtain 503 g of an extract of the aerial part of Aster spathulifolius (EASA).

### Example 2

### Preparation of Administration Forms

### Preparation of Powder

20 mg of the dried powder of the extract of the aerial part of Aster spathulifolius obtained in Example 1 was mixed with 100 mg of lactose and 10 mg of talc. The resultant mixture was filled in an airtight bag to obtain powder.

### Preparation of Tablet

20 mg of the dried powder of the extract of the aerial part of Aster spathulifolius obtained in Example 1 was mixed with 100 mg of cornstarch, 10 mg of lactose and 2 mg of magnesium stearate. The resultant mixture was made into a tablet according to the conventional method.

### Preparation of Capsule

20 mg of the dried powder of the extract of the aerial part of Aster spathulifolius obtained in Example 1 was mixed with 100 mg of cornstarch, 100 mg of lactose and 2 mg of magnesium stearate. The resultant mixture was filled in a gelatin capsule and made into a capsule preparation according to the conventional method.

### Preparation of Solution

To 200 mg of the dried powder of the extract of the aerial part of Aster spathulifolius obtained in Example 1 were added 20 mg of sorbitol, 10 mg of CMC and a little lemon flavor. Then, purified water was added, so that the total volume became 1000 mL. The resultant solution was filled in a brown bottle and sterilized to obtain a solution preparation.

### Test Example 1

### Isolation of Compounds from Extract of Aster spathulifolius

10 L of a filtrate was collected from the first filtration in Example 1. The collected filtrate was concentrated until the volume decreased to 0.5 L using a rotary evaporator at 60° C. and finally freeze-dried to obtain 95 g of an extract of the aerial part of Aster spathulifolius (EASA) in the form of brown-colored powder. Thus-obtained powder was choked in 1 L of methanol and filtered. The mixture was filtered and the filtrate was evaporated to dryness to obtain 17 g of powder. The filtered cake (insoluble material) was also dried to give 76 g, which was composed of sodium chloride and mucous materials. The methanol-soluble material (17 g) was purified by silica gel column chromatography using a mixed solvent (chloroform/methanol/H₂O=5:1:0.1) to obtain 0.98 g of methyl 4,5-dicaffeoylquinate and 0.06 g of 4,5-dicaffeoylquinic acid.

### Compound 1

### Methyl 4,5-dicaffeoylquinate

EI-MS m/z: 530(C₂₆H₂₆O₁₂); [α]²⁵_{D}=−213; IR(KBr)νₘₐₓ(cm⁻¹) 3368, 1701; ¹H-NMR(CDCl₃): 2.08 (1H, dd, J=13.8, 6.6 Hz, 2-CH₂ₑ), 2.32 (1H, dd, J=13.8, 3.6 Hz, 2-CH₂ₐ), 4.34 (1H, ddd, J=6.6, 3.6, 3.3 Hz, 3-CH), 5.01 (1H, dd, J=8.1, 3.3 Hz, 4-CH), 5.53 (1H, dt, J=8.1, 5.4 Hz, 5-CH), 2.18-2.32 (2H, m, 6-CH₂), 3.71 (3H, s, 7-OCH₃), 7.02 (1H, d, J=2.1 Hz, 2′-CH), 7.00 (1H, d, J=2.1 Hz, 2″-CH), 6.75 (2H, d, J=8.4 Hz, 5′-,5″-CH), 6.92 (1H, dd, J=8.4, 2.1 Hz, 6′-CH), 6.91 (1H, dd, J=8.4, 2.1 Hz, 6″-CH), 7.60 (1H, d, J=15.9 Hz, 7′-CH), 7.50 (1H, d, J=15.9 Hz, 7″-CH), 6.29 (1H, d, J=15.9 Hz, 8′-CH), 6.16 (1H, d, J=15.9 Hz, 8″-CH).

### Compound 2

### 4,5-O-Dicaffeoylquinic acid

EI-MS m/z: 516 (C₂₅H₂₄O₁₂); mp 192-194° C.; [α]²⁵_{D}=−170; IR(KBr)νₘₐₓ(cm⁻¹): 3400, 1700, 1610, 1530, 1290, 990, 820; ¹H-NMR (CDCl₃): 2.10 (1H, dd, J=14.4, 4.2 Hz, 2-CH₂ₑ), 2.29 (1H, dd, J=14.4, 3.0 Hz, 2-CH₂ₐ), 4.36 (1H, ddd, J=4.2, 3.3, 3.0 Hz, 3-CH), 5.11 (1H, dd, J=9.0, 3.3 Hz, 4-CH), 5.61 (1H, dt, J=9.0, 5.1 Hz, 5-CH), 2.18-2.23 (2H, m, 6-CH₂), 7.02 (1H, d, J=2.1 Hz, 2′-CH), 7.00 (1H, d, J=2.1 Hz, 2″-CH), 6.73 (1H, d, J=8.4 Hz, 5′-CH), 6.74 (1H, d, J=8.4 Hz, 5″-CH), 6.90 (1H, d, J=8.4, 2.1 Hz, 6′-CH), 6.91 (1H, dd, J=8.4, 2.1 Hz, 6′-CH), 6.91 (1H, dd, J=8.4, 2.1 Hz, 6″-CH), 7.59 (1H, d, J=15.9 Hz, 7′-CH), 7.51 (1H, d, J=15.9 Hz, 7″-CH), 6.28 (1H, d, J=15.9 Hz, 8′-CH), 6.18 (1H, d, J=15.9 Hz, 8″-CH).

### Test Example 2

### Isolation of Active Ingredient from Extract of Aerial Part of Aster spathulifolius

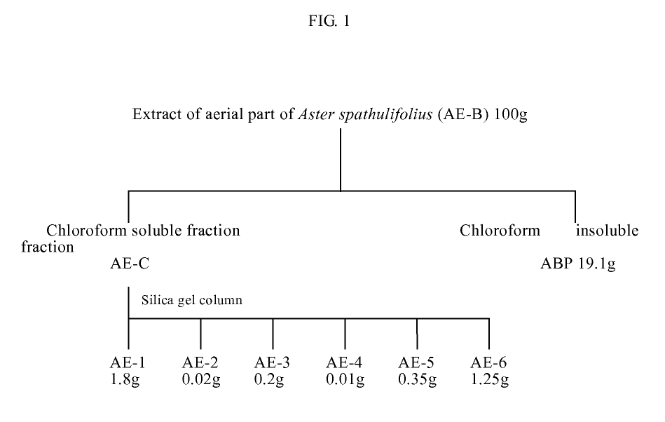

The extract of the aerial part of Aster spathulifolius was suspended in chloroform and kept overnight at room temperature. The suspension was filtered and the chloroform-insoluble portion was 3 times with small portion of chloroform and dried to obtain an insoluble fraction (ABP) and a soluble fraction (AE-C). The combined filtrate was concentrated and subjected to repeated silica gel column chromatography (packed with 200 g of silica gel) using chloroform as eluent to obtain AE-1 (1.8 g), AE-2 (20 mg), AE-3 (200 mg), AE-4 (10 mg) and AE-5 (350 mg), successively (FIG. 1). The column was washed with a mixed solvent (chloroform/methanol=50:1) and the washed solution was concentrated to give ABP (1.25 g) in pure form.

### 2-1. Chemical Structure of ABP (α-Spinasterol 3-O-β-D-glucopyranoide)

Mp: 292-294° C.; EI-MS m/z: 574 (M⁺-C₃₅H₅₈O₆), 395 (M⁺-C₆H₁₁O₆); IR(KBr)νₘₐₓ(cm⁻¹): 3389, 1074, 1032, 970, 829; ¹H-NMR(CDCl₃+CD₃OD)δ: 0.57 (3H, s, 18-CH₃), 0.71 (3H, s, 19-CH₃), 0.84 (3H, d, J=6.3 Hz, 29-CH₃), 0.88 (3H, d, J=7.2 Hz, 26-CH₃), 0.89 (3H, d, J=6.6 Hz), 1.06 (3H, d, J=6.6 Hz, 21-CH₃), 5.02 (1H, d, J=7.5 Hz, anomeric H).

### 2-2. AE-1 (Germacron)

Mp: 51-52° C.; [α]²⁵_{D}−24 (c=1, methanol); EI-MS m/z: 218 (M+, C₁₅H₁₂O); IR(KBr)νₘₐₓ(cm⁻¹): 1672, 1443, 1385, 1289, 1178, 1134, 858, 812; UV(MeOH)λₘₐₓ nm(log ε): 217.5 (4.03); ¹H-NMR(CDCl₃)δ: 4.97 (1H, dd, J=10.5, 10 Hz, 2-CH), 2.35 (1H, m, 3-CH₂), 2.08 (1H, m, 3-CH₂), 2.10 (2H, m, 4-CH₂), 4.70 (1H, ddd, J=10.5, 3.0, 1.0 Hz, 6-CH), 2.90 (2H, m, 7-CH₂), 2.95 (1H, d, 10-CH₂O), 3.40 (1H, d, J=10.5 Hz, 10-CH₂O), 1.76 (3H, s, 12-CH₃), 1.71 (3H, s, 13-CH₃), 1.61 (3H, t-like, 14-CH₃), 1.42 (3H, t-like, 15-CH₃).

### 2-3. Chemical Structure of AE-2 (α/β-Amyrin)

Mp: 168° C.; [α]²⁰_{D}, 8.8 (c=0.2, methanol); EI-MS m/z: 426 (M+, C₃₀H₅₀O); IR(KBr)νₘₐₓ(cm⁻¹): 3293, 1650, 1385, 1359, 1036; ¹H-NMR (CDCl₃)δ: 3.20 (2H, m, 3-CH), 5.13 (1H, t, J=3.3 Hz, α-amyrin [symbol] 12-CH), 5.18 (1H, t, J=3.6 Hz, β-amyrin [symbol] 12-CH).

### 2-4. Chemical Structure of AE-3 (α-Spinasterol)

Mp: 169-170° C.; [α]²⁰_{D}, 15.2 (c=1, methanol); EI-MS m/z: 412 (M+, C₂₉H₄₅O); IR(KBr)νₘₐₓ(cm⁻¹): 3418, 1664, 1041, 970; ¹H-NMR(CDCl₃)δ: 0.54 (3H, s, 18-CH₃), 0.79 (3H, s, 19-CH₃), 1.02 (3H, d, J=6.9 Hz, 21-CH₃), 0.81 (3H, d, J=7.2 Hz, 26-CH₃), 0.84 (3H, d, J=6.3 Hz, 27-CH₃), 0.80 (3H, t, J=6.8 Hz, 29-CH₃).

### 2-5. Chemical Structure of AE-4 (Labda-7,14-dien-13 (R)-ol-β-L-deoxyidopyranoside

Oil-form; [α]²⁰_{D}, −61.0 (c=1, methanol); EI-MS m/z: 436 (M+, C₂₆H₄₄O₅); ¹H-NMR(CD₃OD)δ: 4.79 (1H, d, J=3.6 Hz, 1′-CH), 3.35 (1H, dd, J=6.0, 3.6 Hz, 2′-CH), 3.66 (1H, J=6.0 Hz, 3′-CH), 3.46 (1H, dd, 3=6.0, 3.6 Hz, 4′-CH), 4.23 (1H, dq, J=6.9, 3.6 Hz, 5′-CH), 1.18 (3H, d, J=6.9 Hz, 6′-CH₃).

### 2-6. Chemical Structure of AE-5 (Labda-7,14-dien-13 (R)-ol-β-L-fucopyranoside), New Compound

[α]²⁵_{D}, 8 (c=0.1, methanol); EI-MS m/z: 436 (M+, C₂₆H₄₄O₅); ¹H-NMR(CD₃OD)δ: 0.75 (3H, s, 20-CH₃), 0.85 (3H, s, 18-CH₃), 0.88 (3H, s, 19-CH₃) 1.21 (3H, d, J=6.6 Hz, 6′-CH₃), 1.32 (3H, s, 16-CH₃), 1.66 (3H, s, 17-CH₃), 3.42 (1H, d, J=3.3 Hz, 2′-CH), 3.43 (1H, d, J=1.8 Hz, 3′-CH), 3.50 (1H, dq, J=1.2, 6.6 Hz, 5′-CH), 3.56 (1H, dt, J=1.8, 1.2 Hz, 4′-CH), 4.23 (1H, d, J=7.8 Hz, 1′-CH), 5.16 (1H, dd, J=10.8, 1.2 Hz, 15-CH₂), 5.17 (1H, dd, J=17.7, 1.2 Hz, 15-CH₂), 5.35 (1H, m, 7-CH), 6.04 (1H, dd, J=10.8, 1.2 Hz, 14-CH); ¹³C-NMR(CD₃OD)δ: 39.1 (1-CH₂), 18.5 (2-CH₂), 42.1 (3-CH₂), 32.7 (4-C), 50.3 (5-CH), 23.5 (6-CH₂), 121.6 (7-CH), 135.3 (8-C), 55.5 (9-CH), 37.2 (10-C), 21.2 (11-CH₂), 43.5 (12-CH₂), 80.4 (13-C), 143.0 (14-CH), 113.5 (15-CH₂), 21.7 (16-CH₃), 21.4 (17-CH₃), 32.4 (18-CH₃), 20.9 (19-CH₃), 12.7 (20-CH₃), 98.1 (1′-CH₁), 71.0 (2′-CH), 73.9 (3′-CH), 71.5 (4′-CH), 70.2 (5′-CH), 15.5 (6′-CH₃).

### Test Example 3

### Quantification of Terpene in Extract of the Aerial Part Aster spathulifolius

30 mg of the extract of the aerial part of Aster spathulifolius (AE-B; sample) obtained in Example 1 and 30 mg of α-spinasterol glucopyranoside (ABP; standard) were exactly weighted and dissolved in 40 mL of dimethylsulfoxide (DMSO), respectively. Then, 40% (v/v) ethanol was added to a final volume of 100 mL to obtain each of the sample solution and the standard solution. 80 mg of vanillin was dissolved in 10 mL of ethanol and a 72% (w/w) sulfuric acid solution was prepared.

Total terpene content: Exactly 1 mL was taken from each of the sample solution and the standard solution into a large test tube (diameter 15 mm×length 180 mm). 0.4 mL of the 0.8% vanillin-ethanol solution was added and 5 mL of the 72% sulfuric acid solution, which had been cooled in an ice-water bath, was slowly added to the test tube. Then, the test tube was shaken vigorously. The resultant mixture solution was heated at 60° C. for 60 minutes, then cooled to room temperature and light absorbance was measured at 540 nm. The terpene content of the sample solution was determined using the standard curve of the α-spinasterol glucopyranoside standard solution. A solution prepared by adding 0.4 mL of the 0.8% vanillin-ethanol solution to 1 mL of the sample solution, slowly adding 5 mL of the 72% sulfuric acid solution, which had been cooled in an ice-water bath, to the test tube and shaking the test tube vigorously was used as blank. The total terpene content was calculated by the following equation.

Total terpene content(%)=(Absorbance of sample solution−Absorbance of blank solution)/(Absorbance of standard solution)×100 Equation 1

The analysis result showed that the extract of the aerial part of Aster spathulifolius prepared in Example 1 contained less than 0.1% of salt, which are originally abundant in Aster spathulifolius, less than 0.3% of 4,5-dicaffeoylquinate, which is a phenolic compound having no effect for preventing and treating hyperlipidemia and obesity, and methyl ester thereof and almost non-existent mucous substances. The total content of the terpene compounds, or the active ingredient, including germacron, α- and β-amyrin, α-spinasterol and glycoside thereof, labdadienol, etc. was at least 80.0%.

### Experiment 4

### Animal, Diet, Change of Body Weights and Food Efficiency Ratio

The rats, aged 6 week-old and weighing 170˜180 g Sprague-Dawley male, were purchased from Samtaco (Seoul, Korea). After the animals were given a standard laboratory diet for 1 week, they were divided two groups each group. Rats were fed two diets: the normal diet (ND, n=10) group and high fat diet (HFD, n=50) group. 6 weeks later, HFD group were subdivided five groups. They were administrated with extract of Aster spathulifolius aerial part orally once a day for 6 weeks.

HFD group: High fat diet+0.5% carboxymethycelluose

HFD+AE-B group: High fat diet+AE-B 125 mg/kg

HFD+AE-C group: High fat diet+AE-C 100 mg/kg

HFD+AE-1 group: High fat diet+AE-110 mg/kg

HFD+ABP group: High fat diet+ABP 1.5 mg/kg

The experimental diets contain either a normal fat (11.7% of calories as fat, AIN-76A diet # 100000, Dyets Inc., Bethlehem, Pa., USA) or a high fat (40% of calories as fat, AIN-76A high fat diet # 100496, Dyets Inc., Bethlehem, Pa., USA) (Table 1). During the test period, food intake and body weight were measured twice a week. Food efficiency ratio was calculated by dividing the increase in body weight during the test period by the food intake during the period. The result is given in Table 2

TABLE 1
Composition of experimental diets (g/kg diet)

| Ingredients | Normal diet | High fat diet |
|---|---|---|
| Casein | 200 | 200 |
| DL-Methionine | 3 | 3 |
| Corn starch | 150 | 150 |
| Sucrose | 500 | 345 |
| Cellulose | 50 | 50 |
| Corn Oil | 50 | — |
| Beef Tallow | — | 205 |
| Mineral Mixture | 35 | 35 |
| Vitamin Mixture | 10 | 10 |
| Choline Bitartrate | 2 | 2 |
| Fat % (Calories) | 11.7 | 40.0 |

TABLE 2
[Effects of extract Aster spathulifolius aerial part on body weight, food
intake and food efficiency ratio (PER) of rats fed high fat diet]

| | Food intake | | | | |
|---|---|---|---|---|---|
| Group | (g/day) | 0 wk BW(g) | 6 wk BW(g) | 12 wk B.W(g) | FER |
| ND | 25.26 ± 2.66 | 204.33 ± 3.44 | 422.40 ± 15.32^{a} | 466.20 ± 24.76^{c} | 0.123 ± 0.012^{b} |
| HFD + C | 25.81 ± 2.22 | 205.57 ± 5.44 | 458.40 ± 19.32^{b} | 542.00 ± 26.65^{a} | 0.155 ± 0.012^{a} |
| HFD + AE-B | 25.69 ± 3.12 | 205.57 ± 5.44 | 457.60 ± 18.13^{b} | 504.00 ± 13.49^{b} | 0.137 ± 0.006^{b} |
| HFD + AE-C | 24.69 ± 3.18 | 205.57 ± 5.44 | 458.00 ± 10.35^{b} | 488.80 ± 19.86^{c} | 0.137 ± 0.005^{b} |
| HFD + AE-1 | 25.82 ± 2.91 | 205.57 ± 5.44 | 455.20 ± 15.93^{b} | 504.40 ± 28.37^{b} | 0.137 ± 0.013^{b} |
| HFD + ABP | 26.00 ± 3.64 | 205.57 ± 5.44 | 456.00 ± 21.35^{b} | 509.20 ± 39.54^{ab} | 0.139 ± 0.018^{b} |

After 6 weeks, body weight of ND and HFD-C group are 422.40±15.32 g and 458.40±19.32 g, respectively. And body weight of ND group was significantly decreased compared to those of HFD-C group. After 12 weeks, body weights of those of EASA groups were significantly decreased compared to those of HFD-C group. In Table 2, letters (alphabets) different superscripts are significantly different (p<0.05) among the groups by Duncan's multiple range test.

Change of Light Micrography of White Adipocyte and Adipose Tissue Weights

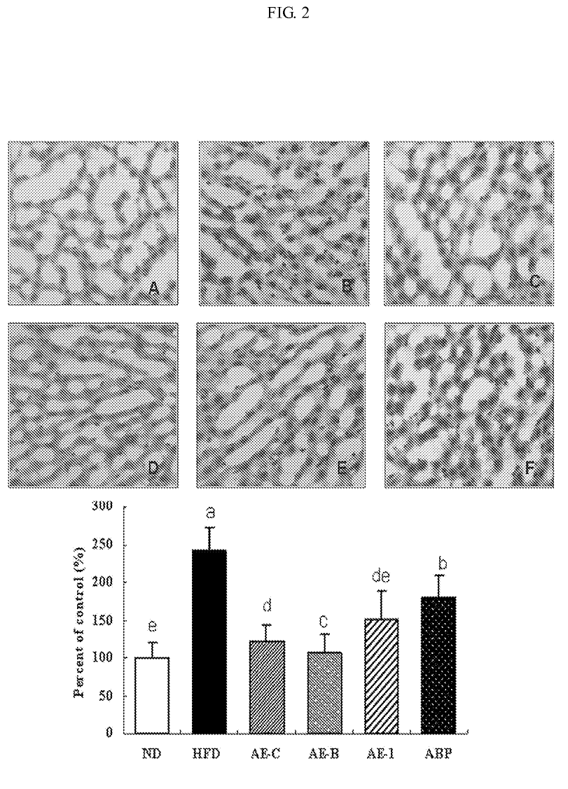

FIG. 2. shows the histological appearance and size of white adipocytes tissue. A is ND Group, B is HFD-C group, C is HFD+AE-B group, D is HFD+AE-C group, E is HFD+AE-1 group and F is HFD+ABP group. The size of adipocytes in ND group were significantly smaller than those of HFD groups. When rats fed EASA, the sizes of adipocytes were quite diminished compared with those of HDF-C.

After 12 weeks, rats were anesthetized with ether and sacrificed after 14 h of fasting. Blood samples were collected from hepatic vein and centrifuged (3,000 rpm for 10 min at 4° C.). Serum was frozen at −70° C. for the biochemical analysis. The epididymal and abdominal fat pad were immediately weighed, measured size of adipocyte using microscope. Weight of adipocyte is shown in Table 3.

The weights of epididymal and abdominal fat in HFD-C group increased more than those of ND and EASA groups (Table 3). There were no differences in epididymal fat weight between ND group and HFD+EASA-C group. EASA were significantly reduced adipocyte size and adipose tissue weight.

TABLE 3

| Group | Epididymal fat pad (g) | Abdominal fat pad (g) |
|---|---|---|
| ND | 8.62 ± 2.85^{b} | 12.01 ± 2.75^{b} |
| HFD + C | 14.32 ± 2.20^{a} | 20.07 ± 2.88^{a} |
| HFD + AE-B | 10.08 ± 3.44^{b} | 14.46 ± 4.28^{b} |
| HFD + AE-C | 8.69 ± 1.87^{b} | 13.21 ± 2.44^{b} |
| HFD + AE-1 | 10.21 ± 2.69^{b} | 14.18 ± 4.67^{b} |
| HFD + ABP | 11.55 ± 1.96^{ab} | 14.98 ± 3.19^{b} |

Measurement of Body Fat with MRI

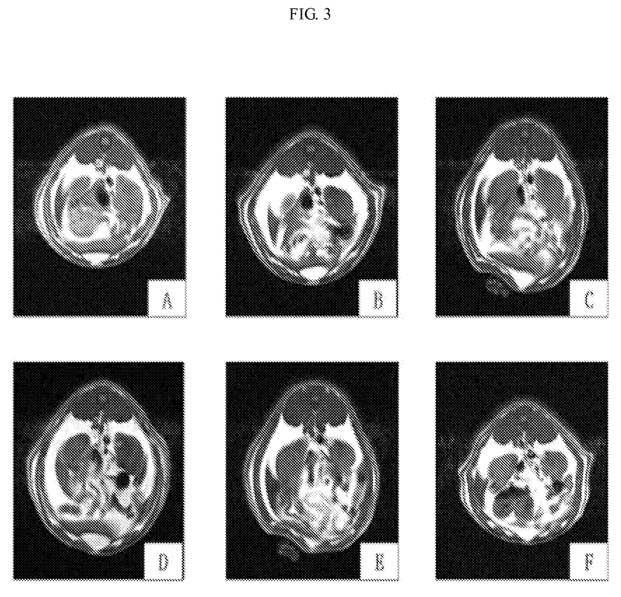

After 12 weeks, the rat was anesthetized with sodium pentobarbital (30 mg/kg, i.p.). Abdominal fat distribution of rats was observed using MRI (Magnetic Resonance Imaging System) on one day before sacrifice (FIG. 3). With MRI, we could visualize large deposits of visceral and subcutaneous fat in HFD rats, but there was little fat present in the abdomen of ND rats. EASA treatment lowered intra-abdomen fat accumulation.

Concentration of Serum Lipids and Liver Lipids

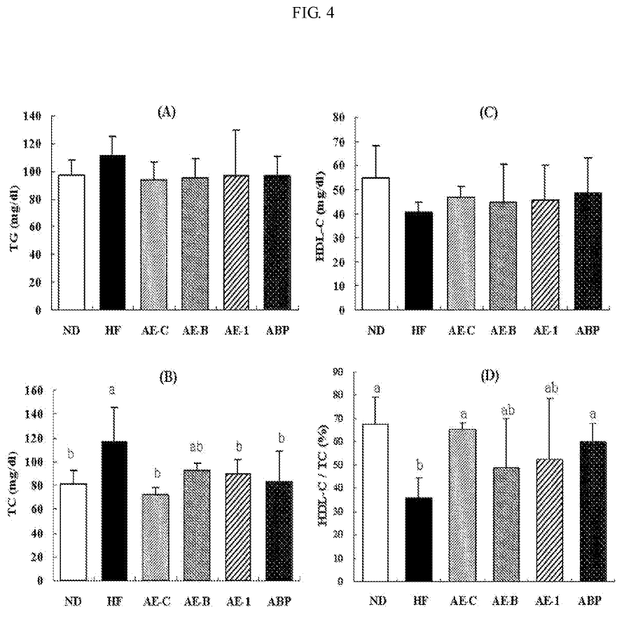

Serum triglyceride (TG), total cholesterol (TC), and HDL-cholesterol (HDL-C) levels were determined using the calorimetric method with commercial kits (Asan Diagnostics, Seoul, Korea). There was no significant difference in serum TG or HDL-C levels, although EASA-treated groups tended to be lower than the HFD-C group (FIG. 4). Serum TC was significantly elevated, by 144.1%, in the HFD-C group compared with the ND group, and significantly reduced by EASA treatment (P<0.05). HFD-C group significantly decreased serum HDL-C/TC levels.

Hepatic lipid was extracted by the method devised by Folch et al. and its content was measured by the same technique used for the serum analysis. The hepatic lipid content depending on the composition of the extract of Aster spathulifolius is given in Table 4. The high fat diet group showed about 2 times larger hepatic triglyceride (TG) level compared with the normal diet group. But, the group to which Aster spathulifolius had been administered showed a noticeable decrease. A similar trend was observed for cholesterol. There was no significant difference in hepatic free fatty acid (FFA) level.

TABLE 4

| Group | TG(mg/g liver) | TC(mg/g liver) | FFA (meq/l) |
|---|---|---|---|
| ND | 1.81 ± 0.48^{b} | 12.99 ± 3.06^{c} | 0.747 ± 0.26 |
| HFD + C | 4.01 ± 1.14^{a} | 20.50 ± 0.76^{a} | 1.076 ± 0.44 |
| HFD + AE-B | 2.40 ± 0.81^{b} | 13.30 ± 2.01^{c} | 1.015 ± 0.19 |
| HFD + AE-C | 2.25 ± 0.10^{b} | 15.35 ± 1.40^{bc} | 0.736 ± 0.15 |
| HFD + AE-1 | 2.20 ± 0.52^{b} | 13.74 ± 0.92^{c} | 0.869 ± 0.25 |
| HFD + ABP | 2.65 ± 0.48^{b} | 17.59 ± 2.79^{b} | 1.018 ± 0.25 |

Concentration of Serum Leptin, Insulin and Glucose

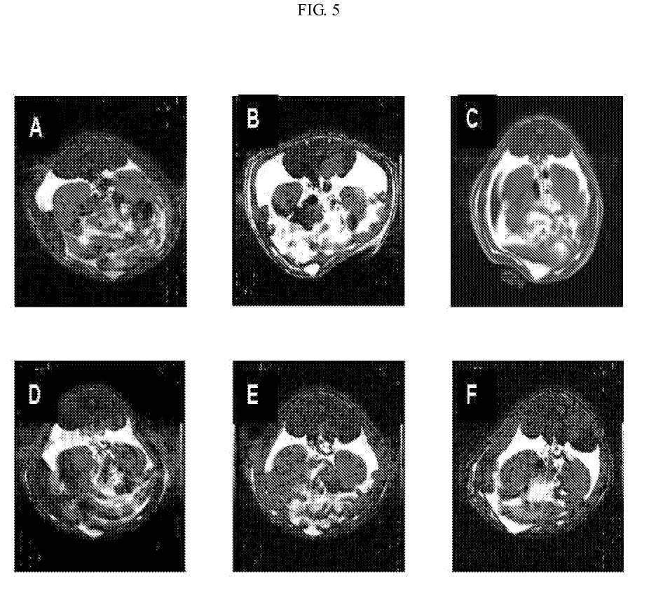

Serum leptin and insulin was measured by RIA using Linco leptin assay kit (Linco research Immunoassay, St. Louis, Mo.) and insulin standards (Linco research Immunoassay, St. Louis, Mo.) respectively (FIG. 5).

TABLE 5
Effects of extract Aster spathulifolius aerial part on serum
leptin, insulin, and glucose level of rats fed high fat diet

| Group | Leptin (ng/ml) | Insulin (ng/ml) | Glucose (mg/dl) |
|---|---|---|---|
| ND | 3.56 ± 0.67^{b} | 0.62 ± 0.15^{c} | 176.60 ± 61.15^{ab} |
| HFD + C | 7.46 ± 1.92^{a} | 1.54 ± 0.55^{a} | 248.60 ± 70.85^{a} |
| HFD + AE-B | 3.90 ± 1.69⁶ | 0.71 ± 0.19^{bc} | 176.40 ± 28.62^{ab} |
| HFD + AE-C | 3.50 ± 0.56^{b} | 0.50 ± 0.13^{c} | 158.80 ± 33.99^{b} |
| HFD + AE-1 | 3.88 ± 1.01^{b} | 1.00 ± 0.27^{bc} | 206.60 ± 42.07^{ab} |
| HFD + ABP | 3.98 ± 0.95^{b} | 1.21 ± 0.60^{bc} | 219.00 ± l3.49^{ab} |

Leptin is produced in adipose tissues, secreted into blood, and delivered to brain through the leptin-receptor to control food intake, appetite, and energy balance, to decrease body weight. A high fat diet increases blood leptin in the mouse, which reflects the increased body fat. Leptin levels are also related to increases in body fat mass and adipocyte size. In our study, serum levels of leptin were over 2 folds higher in the HFD group than in ND group, and EASA group was similar to ND group.

Levels of serum glucose were lower in EASA groups than in HFD group. Especially AE-C and AE-B groups significantly lower than HFD group. Serum levels of insulin were 2.5 folds higher in the HFD group than in ND group but EASA groups significantly decreased more than those of HFD group.

Insulin levels paralleled glucose levels, and were related to leptin levels and body fat mass, putatively because of changes in insulin sensitivity by obesity and blood glucose levels.

Acute Toxicity—Oral Administration

The ICR mouse (weighing 25±5 g) and Sprague-Dawley male (230±10 g) were divided were 4 groups (n=10). Rats were administered with extract of Aster spathulifolius aerial part at 100, 500, 1000, 2000 mg/kg p.o. daily for 14 consecutive days. The control rats were given 1% CMC only. Mice were medicated by gastric infusion respectively with the doses that are 5, 10, 20, 40, 80 and 160 times of clinical dosage and then observed for 22 hours. The results found no mice dead in all groups and change of body weight, and food intake appeared all normal.

### Experiment 5

### Animal, Diet, Change of Body Weights and Food Efficiency Ratio

The rats, aged 6 week-old and weighing 170˜180 g Sprague-Dawley male, were purchased from Samtaco (Seoul, Korea). After the animals were given a standard laboratory diet for 1 week, they were divided two groups each group. Rats were fed two diets: the normal diet (ND, n=8) group and high fat diet (HFD, n=88) group. 6 weeks later, HFD group were subdivided 11 groups (n=8). They were administrated with extract of Aster spathulifolius aerial part orally once a day for 6 weeks.

HFD group: High fat diet

AE-B 250 group: High fat diet+AE-B 250 mg/kg

AE-B 125 group: High fat diet+AE-B 125 mg/kg

AE-B 62.5 group: High fat diet+AE-B 62.5 mg/kg

AE-C 200 group: High fat diet+AE-C 200 mg/kg

AE-C 100 group: High fat diet+AE-C 100 mg/kg

AE-C 50 group: High fat diet+AE-C 50 mg/kg

AE-1 20 group: High fat diet+AE-1 (Germacrone) 20 mg/kg

AE-1 10 group: High fat diet+AE-1(Germacrone) 10 mg/kg

AE-1 5 group: High fat diet+AE-1 (Germacrone) 5 mg/kg

Positive control group: High fat diet+Sibutramine (positive control) 7.5 mg/kg

Experimental diet is same table 1 of experiment 4. Food intake, change of body weights and food efficiency ratio were shown Table 6.

TABLE 6
[Food intake, change of body weights and food efficiency ratio]

| | Food intake | | | | |
|---|---|---|---|---|---|
| Group | (g/day) | 0 wk BW(g) | 6 wk BW(g) | 12 wk B.W(g) | FER |
| ND | 17.65 ± 3.63 | 205.25 ± 6.41 | 384.75 ± 17.38^{b} | 432.75 ± 36.35^{c} | 0.154 ± 0.021^{c} |
| HFD | 18.28 ± 3.35 | 205.25 ± 9.07 | 431.75 ± 38.93^{a} | 549.43 ± 38.64^{a} | 0.223 ± 0.021^{a} |
| Sibutramine | 16.70 ± 2.68 | 205.25 ± 6.41 | 431.25 ± 30.31^{a} | 462.00 ± 44.93^{bc} | 0.183 ± 0.028^{b} |
| AE-B 62.5 | 17.45 ± 2.79 | 205.25 ± 8.07 | 430.50 ± 43.89^{a} | 499.00 ± 47.63^{b} | 0.200 ± 0.029^{ab} |
| AE-B 125 | 17.01 ± 2.49 | 205.25 ± 7.63 | 429.50 ± 33.79^{a} | 484.00 ± 50.05c | 0.194 ± 0.030^{b} |
| AE-B 250 | 17.79 ± 2.99 | 205.50 ± 6.74 | 430.25 ± 32.01^{a} | 474.50 ± 44.96c | 0.180 ± 0.026^{bc} |
| AE-C 50 | 17.06 ± 2.62 | 205.50 ± 6.74 | 429.75 ± 36.07^{a} | 493.00 ± 50.12^{b} | 0.200 ± 0.032^{ab} |
| AE-C 100 | 17.58 ± 3.00 | 205.25 ± 5.85 | 430.25 ± 33.77^{a} | 488.29 ± 51.23^{b} | 0.191 ± 0.032^{b} |
| AE-C 200 | 17.46 ± 2.94 | 205.75 ± 6.36 | 430.00 ± 37.40^{a} | 481.00 ± 43.01^{b} | 0.188 ± 0.025^{b} |
| AE-1 5 | 17.89 ± 2.97 | 205.25 ± 5.75 | 430.25 ± 36.56^{a} | 501.71 ± 46.64^{b} | 0.196 ± 0.029^{b} |
| AE-1 10 | 18.24 ± 2.93 | 205.25 ± 6.41 | 430.00 ± 34.54^{a} | 494.75 ± 45.52^{b} | 0.189 ± 0.026^{b} |
| AE-1 20 | 17.48 ± 2.83 | 205.25 ± 6.41 | 429.25 ± 33.75^{a} | 490.75 ± 18.27^{b} | 0.194 ± 0.010^{b} |

After 12 weeks, Body weights were heavier in the HFD groups than in the ND group. EASAtreated groups reduced body weights versus the HFD group. FER were significantly higher in the HFD group than in the EASAand ND groups. EASA groups were not significantly different from those in the ND group.

Change of Adipocyte Weight

Adipocyte weight were determined method of experiment 5. Adipocyte weight are shown in Table 7. EASA treated groups reduced white adipocyte versus the HFD group, in a dose-dependent manner.

TABLE 7

| Group | Epididymal fat (g) | Abdominal fat (g) | Visceral fat (g) | Total fat (g) |
|---|---|---|---|---|
| ND | 6.36 ± 1.41^{d} | 6.46 ± 2.49^{c} | 4.27 ± 1.29^{c} | 17.10 ± 4.44^{c} |
| HFD | 13.40 ± 2.74^{a} | 17.82 ± 7.09^{a} | 7.48 ± 1.41^{a} | 39.22 ± 9.01^{a} |
| Sibutramine | 8.75 ± 2.57^{bc} | 11.44 ± 3.16^{b} | 5.11 ± 0.89^{b} | 25.30 ± 5.77^{b} |
| AE-B 62.5 | 12.07 ± 3.07^{ab} | 13.31 ± 2.29^{b} | 5.91 ± 1.48^{b} | 31.33 ± 5.03^{b} |
| AE-B 125 | 10.13 ± 2.57^{bc} | 13.02 ± 3.25^{b} | 5.61 ± 0.94^{b} | 28.83 ± 3.59^{b} |
| AE-B 250 | 9.77 ± 1.96^{bc} | 12.21 ± 3.54^{b} | 4.88 ± 0.97^{bc} | 26.85 ± 6.03^{b} |
| AE-C 50 | 11.18 ± 4.08^{ab} | 13.32 ± 3.92^{b} | 5.32 ± 2.08^{b} | 29.82 ± 8.72^{b} |
| AE-C 100 | 10.79 ± 2.57^{ab} | 13.76 ± 1.73^{b} | 4.88 ± 1.19^{b} | 29.43 ± 3.58^{b} |
| AE-C 200 | 9.86 ± 2.55^{b} | 12.46 ± 2.26^{b} | 4.72 ± 0.41^{b} | 25.25 ± 5.53^{b} |
| AE-1 5 | 11.15 ± 2.39^{ab} | 14.50 ± 4.43^{ab} | 5.45 ± 2.13^{b} | 31.09 ± 8.02^{b} |
| AE-1 10 | 9.97 ± 2.85^{b} | 14.60 ± 2.20^{ab} | 4.98 ± 1.53^{b} | 29.55 ± 5.25^{b} |
| AE-1 20 | 11.05 ± 1.44^{b} | 13.76 ± 3.11^{ab} | 4.68 ± 1.78^{b} | 29.49 ± 5.26^{b} |

Measurement of Body Fat with MRI

Weights of epididymal and abdominal fat are shown in Table 5. EASA treated groups reduced the weights of abdominal fat versus the HFD group. In Table 5, A is ND group, B is HFD group, C is Sibutramine group, D is AE-B 250 group, E is AE-C 200 group and F is AE-1 10 group.

Levels of Lipid in Serum and Liver

Levels of TG, TC and HDL-C in serum are shown in Table 8. In levels of TG, the HFD groups had similar values to the ND group. EASA treated groups reduced the levels of TG versus the HFD group. TC was significantly higher in the HFD group than in the ND groups. EASA treated groups reduced the levels of TC versus the HFD and ND group.

TABLE 8

| | Triglyceride | Total cholesterol | HDL-cholesterol |
|---|---|---|---|
| Group | (mg/dl) | (mg/dl) | (mg/dl) |
| ND | 82.00 ± 24.79^{ab} | 82.54 ± 13.43^{b} | 50.17 ± 12.67 |
| HFD | 113.25 ± 36.06^{a} | 100.43 ± 10.70^{a} | 42.57 ± 4.19 |
| Sibutramine | 82.62 ± 26.39^{ab} | 59.79 ± 19.99^{c} | 46.62 ± 12.21 |
| AE-B 62.5 | 97.80 ± 34.70^{ab} | 61.74 ± 8.21^{c} | 45.95 ± 13.30 |
| AE-B 125 | 93.19 ± 24.43^{ab} | 53.34 ± 5.65^{c} | 49.89 ± 10.15 |
| AE-B 250 | 79.93 ± 28.09^{b} | 50.15 ± 16.94^{c} | 51.99 ± 13.67 |
| AE-C 50 | 97.40 ± 28.00^{ab} | 68.83 ± 16.05^{c} | 53.30 ± 16.34 |
| AE-C 100 | 91.91 ± 17.66^{ab} | 59.39 ± 17.34^{c} | 42.70 ± 14.79 |
| AE-C 200 | 81.97 ± 25.97^{ab} | 54.07 ± 15.08^{c} | 56.32 ± 20.50 |
| AE-1 5 | 91.51 ± 20.50^{ab} | 51.93 ± 10.29^{c} | 48.39 ± 9.65 |
| AE-1 10 | 86.94 ± 21.93^{ab} | 62.56 ± 8.33^{c} | 42.41 ± 8.41 |
| AE-1 20 | 91.12 ± 24.03^{ab} | 51.91 ± 14.32^{c} | 55.80 ± 19.57 |

Levels of TG, TC and HDL-C in serum are shown in Table 9. For TG and TC, HFD groups showed a tendency to higher values compared to those with the ED group. EASA treated group showed significantly decreased TC values. Levels of FFA in the HFD group significantly higher than those of the EASA group.

TABLE 9

| Group | TG (mg/g liver) | TC (mg/g liver) | FFA (meq/l) |
|---|---|---|---|
| ND | 1.77 ± 0.51^{b} | 12.33 ± 1.28^{cd} | 0.817 ± 0.40 |
| HFD | 3.78 ± 2.05^{a} | 18.93 ± 0.28^{a} | 1.152 ± 0.43 |
| Sibutramine | 1.84 ± 0.21^{b} | 13.52 ± 2.15^{bc} | 0.871 ± 0.68 |
| AE-B 62.5 | 1.78 ± 0.33^{b} | 12.37 ± 1.92^{cd} | 0.853 ± 0.19 |
| AE-B 125 | 1.73 ± 0.44^{b} | 14.16 ± 1.44^{b} | 0.826 ± 0.36 |
| AE-B 250 | 1.79 ± 0.41^{b} | 11.14 ± 1.30^{d} | 0.741 ± 0.19 |
| AE-C 50 | 1.90 ± 0.43^{b} | 12.48 ± 1.08^{bcd} | 0.835 ± 0.43 |
| AE-C 100 | 1.81 ± 0.52^{b} | 12.19 ± 1.07^{cd} | 0.946 ± 0.40 |
| AE-C 200 | 2.09 ± 0.94^{b} | 12.14 ± 1.97^{cd} | 0.728 ± 0.42 |
| AE-1 5 | 1.95 ± 0.46^{b} | 12.81 ± 1.20^{bcd} | 0.868 ± 0.28 |
| AE-1 10 | 1.81 ± 1.32^{b} | 12.84 ± 1.71^{bcd} | 0.831 ± 0.30 |
| AE-1 20 | 1.88 ± 0.55^{b} | 12.14 ± 1.76^{cd} | 0.775 ± 0.25 |

Serum Levels of Leptin and Insulin

Serum leptin and insulin level was measured in the same manner as in Test Example 4. The result is given in Table 10. Leptin is a hormone secreted from the adipocyte, in proportion to body fat mass. Leptin and insulin are deeply related with energy homeostasis. Increased leptin induces hyperinsulinemia, which in turn directly stimulates the leptin mRNA in the adipocyte. For this reason, although the HFD group showed much higher serum leptin and insulin level than the normal diet group, the group to which the extract of Aster spathulifolius was administered showed a lower level than the HFD group.

TABLE 10

| Group | Leptin (ng/ml) | Insulin (ng/ml) |
|---|---|---|
| ND | 3.50 ± 1.45^{c} | 1.21 ± 0.20^{bc} |
| HFD | 11.60 ± 5.90^{a} | 1.98 ± 0.93^{a} |
| Sibutramine | 6.27 ± 1.89^{bc} | 1.02 ± 0.33^{bc} |
| AE-B 62.5 | 6.93 ± 2.01^{b} | 0.99 ± 0.21^{c} |
| AE-B 125 | 6.71 ± 1.80^{b} | 1.03 ± 0.36^{bc} |
| AE-B 250 | 5.50 ± 2.06^{bc} | 0.88 ± 0.34^{b} |
| AE-C 50 | 6.21 ± 3.22^{bc} | 1.06 ± 0.22^{c} |
| AE-C 100 | 6.24 ± 1.87^{bc} | 0.95 ± 0.16^{bc} |
| AE-C 200 | 5.98 ± 2.07^{bc} | 0.86 ± 0.23^{bc} |
| AE-1 5 | 7.63 ± 1.69^{b} | 1.35 ± 0.28^{bc} |
| AE-1 10 | 6.55 ± 6.28^{bc} | 1.06 ± 0.40^{bc} |
| AE-1 20 | 6.70 ± 2.58^{b} | 0.98 ± 0.21^{b} |

Expression of UCP2 and PPARγ in White Adipose Tissue

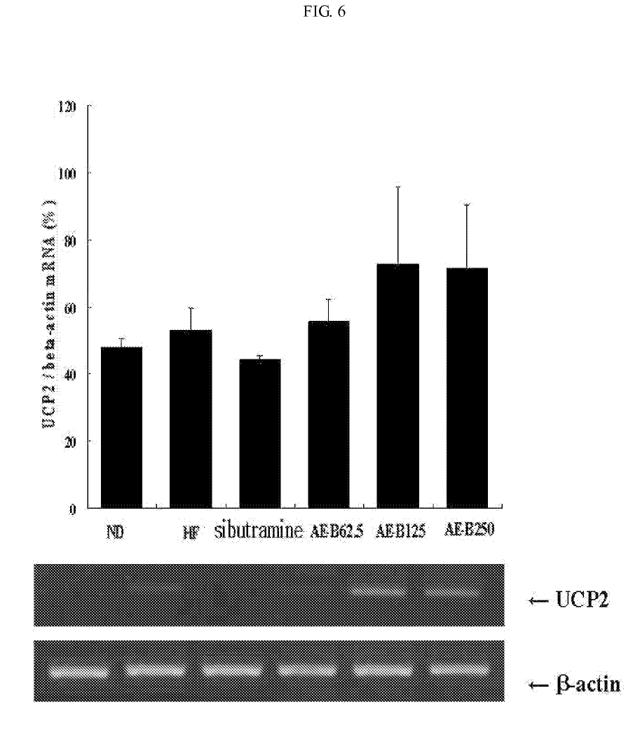

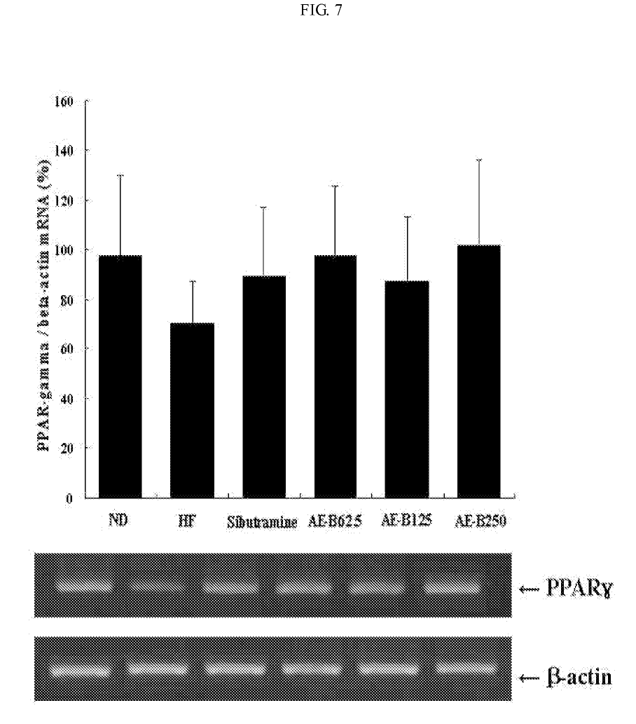

The mRNA levels of several proteins that are involved in the regulation of energy metabolism in white adipose tissue were analyzed. We measured the gene expression of PPARγ and UCP 2 in white adipose tissues (FIGS. 6, 7). As EASA greatly decreased white pad mass, it was sometimes difficult to obtain a sufficient amount of RNA to measure gene expression. The effects of EASAon PPARγ mRNA was investigated from adipose tissue by quantitative RT-PCR. WAT UCP2 mRNA expression was affected by high fatdiet, also, and induced by EASAadministration, dose-dependently

PPARγ is mainly expressed in adipose tissue, and to a lesser extent in colon, the immune system and the retina. UCP are mitochondrial proton transporters that uncouple oxidative phosphorylation by dissipating the proton gradient across the membrane, and this activity has been proposed to influenced thermogenesis and the development of obesity. It is likely that increased expression of UCPs would increase energy expenditure and contribute to the suppression of body fat accumulation. WAT PPARγ mRNA expression was lower by high fat diet, also, and increased by EASAadministration

### INDUSTRIAL APPLICABILITY

The extract of the aerial part Aster spathulifolius in accordance with the present invention has been confirmed to significantly reduce body weight, body fat weight, serum lipid and hepatic lipid level, total cholesterol, free fatty acid level, serum leptin level, insulin, etc. and promote the expression of mRNA's of PPARγ and UCP genes in rats given a high fat diet. Therefore, the extract of the aerial part Aster spathulifolius in accordance with the present invention can be used as functional food for aiding the prevention and treatment of hyperlipidemia, obesity, cardiovascular disease, arteriosclerosis, cerebral thrombosis, liver disease, thrombosis, diabetes, etc. And, since most of salt, which is originally abundant in Aster spathulifolius, phenolic compounds, which are unstable and susceptible to oxidation, and mucous substances are simply removed by the preparation method in accordance with the present invention, the present invention can significantly increase the content of terpenes, or the active ingredient, and reduce production cost.

1-5. (canceled)

6. A composition for preventing and treating hyperlipidemia or obesity comprising at least one of terpene compound selected from the group consisting of germacron, α-spinasterol and glycoside thereof α- and β-amyrin, and labdadienol glycoside.

7. The composition for preventing and treating hyperlipidemia or obesity as set forth in claim 6, wherein the terpene compound is germacron.

8. A functional health food for preventing and improving obesity, hyperlipidemia or cardiovascular disease comprising the extract of the aerial part of Aster spathulifolius.

9. The functional health food as set forth in claim 8, which is in the form of powder, granule, tablet, capsule, suspension, emulsion or syrup.

10. A functional health food for preventing and improving obesity, hyperlipidemia or cardiovascular disease comprising at least one of terpene compound selected from the group consisting of germacron, α-spinasterol and glycoside thereon, α- and β-amyrin, and labdadienol glycoside.

11. A functional health food for preventing and improving obesity, hyperlipidemia or cardiovascular disease as set forth in claim 10, wherein the terpene compound is germacron.

12. (canceled)

13. A composition for preventing and treating hyperlipidemia or obesity comprising the extract of the aerial part of Aster spathulifolius.

14. The composition for preventing and treating hyperlipidemia or obesity as set forth in claim 13, wherein the extract of the aerial part of Aster spathulifolius is obtained by washing the dried powder of the aerial part of Aster spathulifolius with water and drying the washed powder, and then extracting the dried powder in alcohol, a mixture solvent of alcohol and water or a mixture solvent of n-hexane and alcohol.

15. The composition for preventing and treating hyperlipidemia or obesity as set forth in claim 13, wherein the extract of the aerial part of Aster spathulifolius contains 80 wt % to 99.6 wt % of natural terpene compound.

16. The composition for preventing and treating hyperlipidemia or obesity as set forth in claim 15, wherein the natural terpene compound is at least one selected from the group consisting of germacron; α-spinasterol and glycoside thereon; α- and β-amyrin; and labdadienol glycoside.

17. The composition for preventing and treating hyperlipidemia or obesity as set forth in claim 15, wherein the extract of the aerial part of Aster spathulifolius further comprises caffeoylquinate compound in less than 0.3 wt % based on the total weight of the extract of the aerial part of Aster spathulifolius.
